(19) United States
(12) Patent Application Publication
(10) Pub. No.: US 2024/0272311 A1
(43) Pub. Date: Aug. 15, 2024

(54) DETECTOR MODULES FOR SINGLE PHOTON EMISSION COMPUTED TOMOGRAPHY IMAGING

(71) Applicant: UIH AMERICA, INC., Houston, TX (US)

(72) Inventors: Hongdi LI, Houston, TX (US); Zhenrui LU, Shanghai (CN)

(73) Assignee: UIH AMERICA, INC., Houston, TX (US)

(21) Appl. No.: 18/169,871

(22) Filed: Feb. 15, 2023

(51) Int. Cl. G01T 1/20; G01T 1/202; G06N 20/00

(52) U.S. Cl. CPC G01T 1/2018; G01T 1/202; G06N 20/00

(57) ABSTRACT

A detector module for single SPECT may be provided. The detector module may include a semi-monolithic crystal and a plurality of SiPM photodetectors forming a photodetector array. The semi-monolithic crystal may include a plurality of monolithic crystal plates configured to receive gamma rays. The plurality of monolithic crystal plates may be arranged side by side along a thickness direction of the plurality of monolithic crystal plates. The photodetector array may include a plurality of columns arranged side by side along the thickness direction of the plurality of monolithic crystal plates. Each monolithic crystal plate may be in optical communication with one or more columns of SiPM photodetectors in the photodetector array, and the one or more columns of SiPM photodetectors may be configured to detect scintillation light produced by gamma ray interactions in the monolithic crystal plate.

### TECHNICAL FIELD

The present disclosure generally relates to single photon emission computed tomography (SPECT) imaging, and in particular, to systems and methods for detector modules for SPECT imaging.

### BACKGROUND

SPECT imaging is one of the nuclear medicine functional imaging techniques widely used in medical diagnosis (e.g., diagnosis of prostate cancer, neuroendocrine tumors, neuroblastoma, pheochromocytoma, or other diseases), preclinical scientific research, new drug development, etc. For example, a SPECT image may be indicative of some physiological parameters of tracer kinetics and can aid the evaluation of the physiology (or functionality) and/or anatomy (or structure) of a target organ or tissue, as well as its biochemical properties.

### SUMMARY

According to an aspect of the present disclosure, a detector module for SPECT may be provided. The detector module may include a semi-monolithic crystal and a plurality of silicon photomultiplier (SiPM) photodetectors. The semi-monolithic crystal may include a plurality of monolithic crystal plates configured to receive gamma rays. The plurality of monolithic crystal plates may be arranged side by side along a thickness direction of the plurality of monolithic crystal plates. The plurality of SiPM photodetectors may form a photodetector array. The photodetector array may include a plurality of columns arranged side by side along the thickness direction of the plurality of monolithic crystal plates. For each of the plurality of monolithic crystal plates, the monolithic crystal plate may be in optical communication with one or more columns of SiPM photodetectors in the photodetector array, and the one or more columns of SiPM photodetectors may be configured to detect scintillation light produced by gamma ray interactions in the monolithic crystal plate.

In some embodiments, the detector module may be electronically connected to a processing device, and the processing device may be configured to perform the following operations. The processing device may receive readout signals from one or more target columns of SiPM photodetectors in the photodetector array that are in optical communication with a target monolithic crystal plate among the plurality of monolithic crystal plates. The processing device may further determine that a target gamma ray interaction occurs in the target monolithic crystal plate based on the readout signals.

In some embodiments, the one or more target columns of SiPM photodetectors include a plurality of rows arranged along a length direction of the target monolithic crystal plate, and the processing device may be further configured to perform the following operations. The processing device may determine a total signal intensity detected by each row of the plurality of rows based on the readout signals. The processing device may further determine position information of the target gamma ray interaction in the target monolithic crystal plate based on the total signal intensity received by each row of the plurality of rows.

In some embodiments, to determine position information of the target gamma ray interaction in the target monolithic crystal plate based on the total signal intensity received by each row of the plurality of rows, the processing device may be configured to perform the following operations. The processing device may determine a signal intensity distribution in the plurality of rows based on the total signal intensity received by each row of the plurality of rows. The processing device may further determine at least one of first position information or second position information based on the signal intensity distribution. The first position information may relate to the position of the target gamma ray interaction along the length direction of the target monolithic crystal plate, and the second position information may relate to the position of the target gamma ray interaction along a depth direction of the target monolithic crystal plate.

In some embodiments, to determine at least one of first position information or second position information based on the signal intensity distribution, the processing device may be configured to determine the at least one of the first position information or the second position information by processing the signal intensity distribution using a position information determination model.

In some embodiments, the position information determination model may be generated by a model training process. A plurality of training samples may be obtained. Each training sample may include a sample signal intensity distribution corresponding to a sample gamma ray interaction and reference position information of the sample gamma ray interaction. The position information determination model may be generated by training a preliminary model using the plurality of training samples.

In some embodiments, the detector module may further include a light guide disposed between the semi-monolithic crystal and the photodetector array, and configured to guide the scintillation light from the semi-monolithic crystal to the photodetector array.

In some embodiments, a material of the semi-monolithic crystal may include at least one of cesium iodide (CsI) or sodium iodide (NaI).

In some embodiments, a thickness of each of at least a portion of the plurality of monolithic crystal plates may be smaller than 1.3 millimeters.

In some embodiments, a distance between adjacent monolithic crystal plates among the plurality of monolithic crystal plates may be smaller than 0.1 millimeters.

In some embodiments, a size of each column in the photodetector array along the thickness direction of the plurality of monolithic crystal plates may be greater than a thickness of the monolithic crystal plate being in optical communication with the column.

In some embodiments, a signal readout sampling rate of each of the plurality of SiPM photodetectors may be in a range from 20 MHz to 150 MHz.

In some embodiments, a range of energy that the detector module focuses on may be a range from 20 kev to 1000 kev.

According to another aspect of the present disclosure, a SPECT device may be provided. The SPECT device may include a detector module and a collimator. The collimator may be configured to limit a range of photons entering the detector module. The detector module may be configured to detect photons. The detector module may include a semi-monolithic crystal and a plurality of silicon photomultiplier (SiPM) photodetectors. The semi-monolithic crystal may include a plurality of monolithic crystal plates configured to receive gamma rays. The plurality of monolithic crystal plates may be arranged side by side along a thickness direction of the plurality of monolithic crystal plates. The plurality of SiPM photodetectors may form a photodetector array. The photodetector array may include a plurality of columns arranged side by side along the thickness direction of the plurality of monolithic crystal plates. For each of the plurality of monolithic crystal plates, the monolithic crystal plate may be in optical communication with one or more columns of SiPM photodetectors in the photodetector array, and the one or more columns of SiPM photodetectors may be configured to detect scintillation light produced by gamma ray interactions in the monolithic crystal plate.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a block diagram illustrating an exemplary SPECT imaging system according to some embodiments of the present disclosure;

FIG. 2 is a schematic diagram illustrating an exemplary detector module for SPECT according to some embodiments of the present disclosure;

FIGS. 3A-3C illustrates side views of exemplary detector modules according to some embodiments of the present disclosure;

FIG. 4 is a block diagram illustrating an exemplary processing device according to some embodiments of the present disclosure;

FIG. 5 is a flowchart illustrating an exemplary process for determining position information of a target gamma ray interaction according to some embodiments of the present disclosure;

FIG. 6 provides a schematic diagram illustrating exemplary reference gamma ray interactions and their respective reference signal intensity curves according to some embodiments of the present disclosure; and

FIG. 7 is a flowchart illustrating an exemplary process for generating a position information determination model according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections, or assemblies of different levels in ascending order. However, the terms may be displaced by another expression if they achieve the same purpose.

Generally, the words “module,” “unit,” or “block” used herein refer to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for performing on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to performing). Such software code may be stored, partially or fully, on a storage device of the performing computing device, for performing by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments of the present disclosure. It is to be expressly understood, the operations of the flowcharts may be implemented not in order. Conversely, the operations may be implemented in inverted order, or simultaneously. Moreover, one or more other operations may be added to the flowcharts. One or more operations may be removed from the flowcharts.

The term “image” in the present disclosure is used to collectively refer to imaging data (e.g., scan data, projection data) and/or images of various forms, including a two-dimensional (2D) image, a three-dimensional (3D) image, a four-dimensional (4D), etc. The term “pixel” and “voxel” in the present disclosure are used interchangeably to refer to an element of an image. The term “region,” “location,” and “area” in the present disclosure may refer to a location of an anatomical structure shown in the image or an actual location of the anatomical structure existing in or on a target object's body, since the image may indicate the actual location of a certain anatomical structure existing in or on the target object's body.

Conventionally, a detector module of a SPECT scanner includes an analog Anger detector module or a Cadmium zinc telluride (CZT) detector module. The analog Anger detector module includes a photomultiplier (PMT) photodetector and a monolithic scintillator. Due to a physical size limitation of the PMT photodetector and the use of the monolithic scintillator which results in a relatively large propagation range of scintillation photons, a spatial resolution of the analog Anger detector module is low. The CZT detector module has a compact and lightweight spatial structure, a high spatial resolution (e.g., a spatial resolution of 2.46 mm) and energy resolution, and a high scanning efficiency. However, the CZT detector module is usually used for high-energy quantitative detection, which may cause relatively great quantitative errors due to distortion. In addition, the cost of CZT detector module is relatively high.

An improved detector module for SPECT is needed to address the above-mentioned problems of conventional detector modules. One of the promising approaches is using silicon photomultiplier (SiPM) photodetectors. A size of a SiPM photodetector may be a millimeter-level. Conventional PMT photodetector normally has a size in a centimeter-level. Therefore, the size of the SiPM photodetector is much smaller than the size of the PMT photodetector. Therefore, at the same spatial resolution level, the detector module including the SiPM photodetectors has a much smaller size than a conventional detector module using PMT photodetectors. SiPM photodetectors have been widely used for positron emission tomography (PET) imaging. However, the SiPM photodetectors have not been used for SPECT imaging due to some reasons. For example, if the SiPM photodetectors are used for SPECT imaging, dark currents occur in the SiPM photodetectors will result in that an energy output signal (a sum of all signals detected by the SiPM photodetectors) of the detector module in the SPECT scanner has a non-negligible background noise, which reduces a signal-to-noise ratio of a resulting SPECT image and an energy resolution of the SiPM photodetectors. The reasons for the above problems may include: 1) In PET imaging, coincidence events need to be determined based on a nanosecond-level time window, for example, a coincidence event may be recorded when a pair of photons generated by a positron-electron annihilation are detected within a coincidence time window, e.g., within 6 to 12 nanoseconds. Since noises generated by the SiPM photodetectors occur randomly, the noises generated by the SiPM photodetectors may be eliminated after the coincidence event processing, so the background noise generated by the dark currents in the SiPM photodetectors does not affect PET imaging. However, there is no coincidence event processing in SPECT imaging, all events entering the SiPM photodetectors can be received, the background noise due to dark currents generated by the SiPM photodetectors will affect SPECT imaging; 2) SPECT imaging needs to focus on a wide range of energy (e.g., a range from 20 kev to 1000 kev), while PET imaging only focuses on a single 511 kev energy, so the dark current in the SiPM photodetectors has a greater impact on the energy resolution of SPECT imaging, especially the resolution of characteristic peaks with low energy; 3) In SPECT imaging, relatively few photons enter the detector module after passing through a collimator, so the background noise generated by the SiPM photodetectors can have a greater impact on SPECT imaging. Therefore, it is desirable to provide a detector module using SiPM photodetectors with a negligible background noise.

According to one aspect of the present disclosure, a detector module for SPECT may be provided. The detector module may include a semi-monolithic crystal and a plurality of SiPM photodetectors forming a photodetector array. The semi-monolithic crystal may include a plurality of monolithic crystal plates configured to receive gamma rays emitted by a radioactive tracer injected into a subject, and the plurality of monolithic crystal plates may be arranged side by side along a thickness direction of the plurality of monolithic crystal plates. The photodetector array may include a plurality of columns arranged side by side along the thickness direction of the plurality of monolithic crystal plates. For each of the plurality of monolithic crystal plates, the monolithic crystal plate may be in optical communication with one or more columns of SiPM photodetectors in the photodetector array, and the one or more columns of SiPM photodetectors may be configured to detect scintillation light produced by gamma ray interactions in the monolithic crystal plate. According to some embodiments of the present disclosure, the crystal in the detector module may be the semi-monolithic crystal including the plurality of monolithic crystal plates, which is different from the conventional monolithic scintillator. The scintillation light produced by gamma ray interactions in a specific monolithic crystal plate can only travel in the specific monolithic crystal plate and be detected by SiPM photodetectors being in optical communication with the specific monolithic crystal plate. In this way, only a small number of SiPM photodetectors are capable of detecting the scintillation light, the background noise caused by the dark currents in the small number of SiPM photodetectors is relatively small, thereby ensuring the signal-to-noise ratio of a resulting SPECT image and the energy resolution of the SiPM photodetectors, that is, the detector module of the present disclosure can effectively solve the problems caused by the use of SiPM photodetectors in SPECT imaging.

Compared with the conventional analog Anger detector module, the detector module of the present disclosure may have an improved spatial resolution by using the semi-monolithic crystal and the SiPM photodetectors with a size smaller than the PMT photodetectors.

Compared with the conventional CZT detector module, the cost of the detector module of the present disclosure is lower and the spatial resolution is higher.

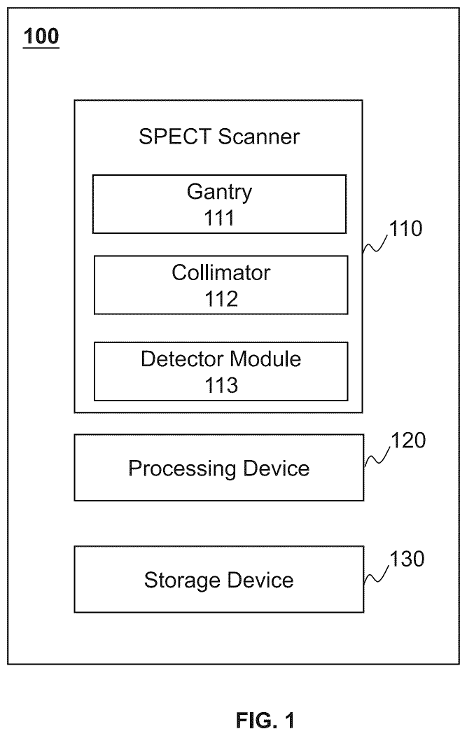

FIG. 1 is a block diagram illustrating an exemplary SPECT imaging system 100 according to some embodiments of the present disclosure. As illustrated in FIG. 1, the SPECT imaging system 100 may include a SPECT scanner 110, a processing device 120, and a storage device 130. The components in the SPECT imaging system 100 may be connected in various ways. Merely by way of example, the SPECT scanner 110 may be connected to the processing device 120 through a network. As another example, the SPECT scanner 110 may be connected to the processing device 120 directly.

The SPECT scanner 110 may be configured to acquire scan data relating to an object. For example, the SPECT scanner 110 may scan the object or a portion thereof that is located within its detection region and generate scan data relating to the object or the portion thereof. The object may be a biological object (e.g., a patient, an animal) or a non-biological object (e.g., a phantom). In some embodiments, the object may include a specific part, organ, and/or tissue of the object. For example, the object may include the head, the bladder, the brain, the neck, the torso, a shoulder, an arm, the thorax, the heart, the stomach, a blood vessel, soft tissue, a knee, a foot, or the like, or any combination thereof, of a patient. In the present disclosure, “object” and “subject” are used interchangeably.

Before a scan performed by the SPECT scanner 110, the object may be injected with a radioactive tracer. For example, the object may be scanned by the SPECT scanner 110 in a predetermined time period after the radioactive tracer is injected into the object. As another example, the object may be scanned by the SPECT scanner 110 in a certain time period after the radioactive tracer distribution in the object reaches equilibrium or steady-state. In some embodiments, the radioactive tracer may include technetium-99 (Tc-99), fluorine-18 (F-18), indium-111 (In-111), iodine-131 (I-131), or the like, or any combination thereof.

In some embodiments, the SPECT scanner 110 may include a single modality scanner or a multi-modality imaging device. For example, the SPECT scanner 110 may include a SPECT device, a SPECT-CT device, a SPECT-PET device, a SPECT-MR device, etc.

In some embodiments, the SPECT scanner 110 may include a gantry 111, a collimator 112, a detector module 113, and/or other components not shown. The gantry 111 may support one or more parts of the SPECT scanner 110, for example, the collimator 112, the detector module 113, and/or other components. The collimator 112 may limit a range of photons (e.g., γ photons) entering the detector module 113. In some embodiments, the collimator 112 may be a multi-pinhole collimator having at least two sets of pinholes. Each set of pinholes may include one or more pinholes.

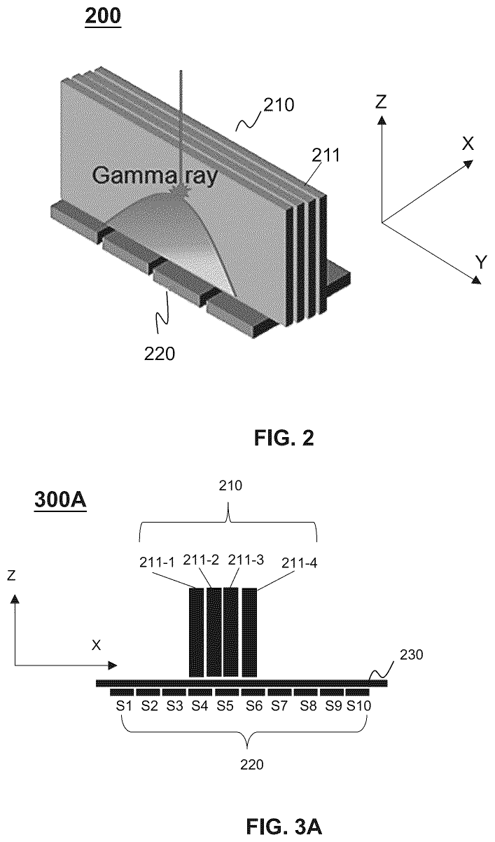

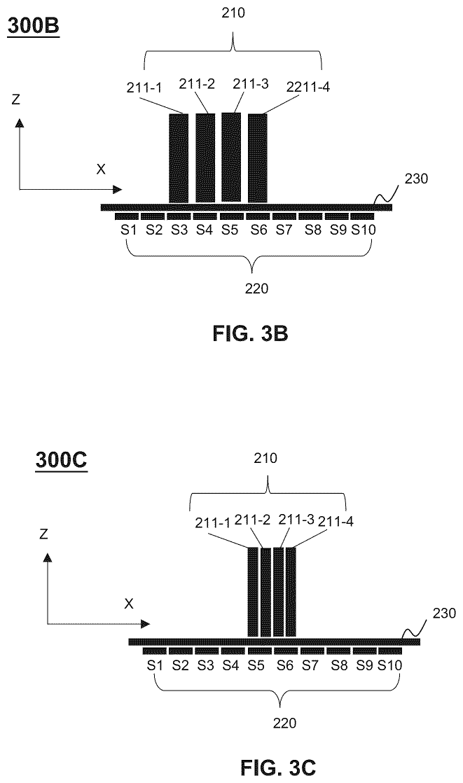

The detector module 113 may be configured to detect the photons collimated by the collimator and generate electrical signals. The detector module 113 may include a semi-monolithic crystal and a plurality of SiPM photodetectors. The semi-monolithic crystal may be configured to receive gamma rays emitted by the radioactive tracer injected into the subject. The SiPM photodetectors may form a photodetector array may be configured to detect scintillation light produced by gamma ray interactions in the semi-monolithic crystal. In some embodiments, the detector module 113 may have a relatively small size and be mounted on a specific position of the detection tunnel of the gantry 111. In the SPECT scan, the gantry 111 may rotate around the object being scanned, and the detector module 113 may rotate with the gantry 111 to detect photons from different perspectives. In some embodiments, the detector module 113 may have a relatively big size that wraps around the detection tunnel of the gantry 111. In the SPECT scan, the gantry 111 and the detector module 113 may remain still. More descriptions for the detector module 113 may be found elsewhere in the present disclosure (e.g., FIGS. 2-3C and the descriptions thereof).

The processing device 120 may process data obtained from one or more components (e.g., the SPECT scanner 110, or the storage device 130) of the SPECT system 100. For example, the processing device 120 may be electronically connected to the SPECT scanner 110. The processing device 120 may receive readout signals from the photodetector array. Further, the processing device 120 may determine position information of a target gamma ray interaction in the semi-monolithic crystal based on the readout signals.

In some embodiments, the processing device 120 may be a single server or a server group. The server group may be centralized or distributed. In some embodiments, the processing device 120 may be local or remote. Merely for illustration, only one processing device 120 is described in the SPECT imaging system 100. However, it should be noted that the SPECT imaging system 100 in the present disclosure may also include multiple processing devices. Thus operations and/or method steps that are performed by one processing device 120 as described in the present disclosure may also be jointly or separately performed by the multiple processing devices. For example, if in the present disclosure the processing device 120 of the SPECT imaging system 100 executes both process A and process B, it should be understood that the process A and the process B may also be performed by two or more different processing devices jointly or separately in the SPECT imaging system 100 (e.g., a first processing device executes process A and a second processing device executes process B, or the first and second processing devices jointly execute processes A and B).

The storage device 130 may store data and/or instructions. In some embodiments, the storage device 130 may store data obtained from the SPECT scanner 110 and/or the processing device 120. In some embodiments, the storage device 130 may store data and/or instructions that the processing device 120 may execute or use to perform exemplary methods described in the present disclosure. In some embodiments, the storage device 130 may include a mass storage, removable storage, a volatile read-and-write memory, a read-only memory (ROM), or the like, or any combination thereof. In some embodiments, the storage device 130 may be implemented on the cloud platform described elsewhere in the present disclosure.

This description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. However, those variations and modifications do not depart the scope of the present disclosure. Merely by way of example, the SPECT imaging system 100 may include one or more additional components and/or one or more components described above may be omitted. For example, the SPECT imaging system 100 may include a network. The network may include any suitable network that can facilitate the exchange of information and/or data for the SPECT imaging system 100. In some embodiments, one or more components of the SPECT imaging system 100 (e.g., the SPECT scanner 110, the processing device 120, etc.) may communicate information and/or data with one or more other components of the SPECT imaging system 100 via the network.

FIG. 2 is a schematic diagram illustrating an exemplary detector module 200 for SPECT according to some embodiments of the present disclosure. In some embodiments, the detector module 200 may be an exemplary embodiment of the detector module 113 of the SPECT scanner 110 as described in FIG. 1.

The detector module 200 may be configured to detect photons. In some embodiments, a range of energy that the detector module focuses on may be a range from 20 kev to 1000 kev. In some embodiments, the range of energy that the detector module focuses on is related to a radioactive tracer for SPECT scan. For example, if the radioactive tracer is iodine-131 (I-131), the range of energy that the detector module focuses on may be 364 keV. As another example, if the radioactive tracer is lutecium-177 (Lu-177), the range of energy that the detector module focuses on may be 208 keV.

As shown in FIG. 2, the detector module 200 may include a semi-monolithic crystal 210 and a photodetector array 220. The photodetector array 220 may be formed by a plurality of SiPM photodetectors.

As used herein, a semi-monolithic crystal refers a crystal that is not monolithic in at least one direction and is monolithic in one or more directions other than the at least one direction. For example, at least a portion of a monolithic crystal may be divided into multiple crystal plates arranged along the at least one direction to form the semi-monolithic crystal. As shown in FIG. 2, the semi-monolithic crystal 210 may include a plurality of monolithic crystal plates 211. A monolithic crystal plate 211 refers to a crystal whose one side is much shorter than other sides. For example, the monolithic crystal plate 211 may be a cuboid with one side much shorter than the other two sides. The plurality of monolithic crystal plates 211 may be configured to receive gamma rays emitted by a radioactive tracer injected into a subject. The plurality of monolithic crystal plates 211 may be arranged side by side along a thickness direction of the plurality of monolithic crystal plates 211.

As used herein, a surface of a monolithic crystal plate 211 that receives gamma rays from the subject is referred to as an incident plane; the thickness direction of the monolithic crystal plate 211 refers to a direction along which the short side of the incident plane extends; the length direction of the monolithic crystal plate 211 refers to a direction along which the long side of the incident plane extends; and the depth direction of the monolithic crystal plate 211 refers to a direction along which a side of the monolithic crystal plate 211 perpendicular to the incident plane extends. For example, referring to FIG. 2, the top surface of a monolithic crystal plate 211 is referred to as an incident plane, the thickness direction is parallel to an X-axis direction, the length direction is parallel to a Y-axis direction, and the depth direction is parallel to a Z-axis direction.

In some embodiments, the plurality of monolithic crystal plates 211 may include a plurality of columns along the thickness direction (i.e., the X-axis direction in FIG. 2) and a plurality of rows along a length direction (i.e., the Y-axis direction in FIG. 2) of the plurality of monolithic crystal plates 211.

In some embodiments, a material of the semi-monolithic crystal 210 may include cesium iodide (CsI), sodium iodide (NaI), or the like, or any combination thereof. Since NaI is prone to deliquescence, the semi-monolithic crystal 210 may be sealed as soon as possible after the semi-monolithic crystal 210 is made using NaI.

In some embodiments, a length, a depth, and/or a thickness of each of at least a portion of the plurality of monolithic crystal plates 211 may be set according to a default setting of the SPECT imaging system 100 or an actual need. For example, the length of each of at least a portion of the plurality of monolithic crystal plates 211 may be greater than 10 millimeters (e.g., 15 millimeters, 20 millimeters, etc.). As another example, the depth of each of at least a portion of the plurality of monolithic crystal plates 211 may be greater than 5 millimeters (e.g., 8 millimeters, 10 millimeters, etc.). As still another example, the thickness of each of at least a portion of the plurality of monolithic crystal plates 211 may be smaller than 2 millimeters (e.g., 1.5 millimeters, 1.3 millimeters, etc.). In some embodiments, the smaller the thicknesses of the plurality of monolithic crystal plates 211 are, the greater the spatial resolution of the detector module 200 may be. In some embodiments, the thickness of each of at least a portion of the plurality of monolithic crystal plates 211 may be smaller than 1.3 millimeters.

In some embodiments, a distance between adjacent monolithic crystal plates may be set according to a default setting of the SPECT imaging system 100 or an actual need. For example, the distance between adjacent monolithic crystal plates 211 among the plurality of monolithic crystal plates 211 may be smaller than 0.5 millimeters (e.g., 0.2 millimeters, 0.1 millimeters, etc.). In some embodiments, the smaller the distance between adjacent monolithic crystal plates 211 among the plurality of monolithic crystal plates 211 is, the greater a sensitivity of the detector module 200 may be. In some embodiments, the distance between adjacent monolithic crystal plates 211 among the plurality of monolithic crystal plates 211 may be smaller than 0.1 millimeters. In some embodiments, the distance between adjacent monolithic crystal plates 211 may be set based on a separation layer arranged between adjacent monolithic crystal plates 211.

The photodetector array 220 may include a plurality of columns arranged side by side along the thickness direction of the plurality of monolithic crystal plates 211. For each monolithic crystal plate 211, the monolithic crystal plate 211 may be in optical communication with one or more columns of SiPM photodetectors in the photodetector array 220, and the one or more columns of SiPM photodetectors may be configured to detect scintillation light produced by gamma ray interactions in the monolithic crystal plate 211. For example, each of some monolithic crystal plates 211 may be in optical communication with one column of SiPM photodetectors. As another example, each of some monolithic crystal plates 211 may be in optical communication with at least two columns of SiPM photodetectors. As still another example, a plurality of monolithic crystal plates 211 may be in optical communication with a same column of SiPM photodetectors.

Merely by way of example, FIGS. 3A-3C illustrates side views of exemplary detector modules 300A, 300B, and 300C according to some embodiments of the present disclosure. The side views shown in FIGS. 3A-3C are seen from a direction along the length direction of the monolithic crystal plates 211 (i.e., the Y direction). The detector modules 300A, 300B, and 300C may be exemplary embodiments of the detector module 200. As shown in FIG. 3A-3C, the semi-monolithic crystal 210 may include four monolithic crystal plates 211-1, 211-2, 211-3, and 211-4. The photodetector array 220 may include ten columns S1-S10 of SiPM photodetectors.

As shown in FIG. 3A, the monolithic crystal plates 211-1, 211-3, and 211-4 may be in optical communication with the columns S4, S5, and S6 of SiPM photodetectors, respectively. The monolithic crystal plate 211-2 may be in optical communication with both the columns S4 and S5 of SiPM photodetectors. As shown in FIG. 3B, each monolithic crystal plate may be in optical communication with one column of SiPM photodetectors, that is, the monolithic crystal plates 211-1, 211-2, 211-3, and 211-4 may be in optical communication with the columns S3, S4, S5, and S6 of SiPM photodetectors, respectively. As shown in FIG. 3C, the monolithic crystal plates 211-1 and 211-2 may both be in optical communication with the column S5 of SiPM photodetectors. The monolithic crystal plates 211-3 and 211-4 may both be in optical communication with the column S6 of SiPM photodetectors.

Different connections between the monolithic crystal plates 211 and the columns of SiPM photodetectors may have different influences on the spatial resolution of the detector module 200 and subsequent determination of gamma ray interactions in the monolithic crystal plates 211. If a plurality of monolithic crystal plates 211 are in optical communicate with only one column of SiPM photodetectors, the specific monolithic crystal plate where the gamma ray interaction occurs is not located based on readout signals from the columns of SiPM photodetectors, and in this connection manner, the spatial resolution of the detector module 200 may be reduced. In contrast, in other connection manners, the specific monolithic crystal plate where the gamma ray interaction occurs may be located based on readout signals from the columns of SiPM photodetectors. The connections between the monolithic crystal plates 211 and the columns of SiPM photodetectors may be determined according to actual requirements. Preferably, different columns of SiPM photodetectors may be in optical communicate with different monolithic crystal plates 211 and one column of SiPM photodetectors may be in optical communicate with only one monolithic crystal plate 211, which may quickly locate the monolithic crystal plate where the gamma ray interaction occurs.

In some embodiments, a size of each column in the photodetector array 220 along the thickness direction of the plurality of monolithic crystal plates 211 may be greater than the thickness of the monolithic crystal plate being in optical communication with the column to facilitate the detection of the scintillation light.

In some embodiments, as shown in FIG. 2, the photodetector array 220 may include a plurality of rows arranged side by side along the length direction (i.e., the Y-axis direction in FIG. 2) of the plurality of monolithic crystal plates 211, and each monolithic crystal plate 211 may correspond to the plurality of rows, which may facilitate to determine the position information of the gamma ray interaction along the length direction of the monolithic crystal plate described in FIG. 5.

In some embodiments, at least one of a thickness, a length, or a height of each of the plurality of SiPM photodetectors may be smaller than 1 centimeter, that is, a size of each of the plurality of SiPM photodetectors may be a millimeter-level. Conventional PMT photodetector normally has a size in a centimeter-level. Therefore, the size of a SiPM photodetector is much smaller than the size of a PMT photodetector. Therefore, at the same spatial resolution level, the detector module 200 including the SiPM photodetectors has a much smaller size than a conventional detector module using PMT photodetectors (e.g., an analog Anger detector module). For example, a conventional ring detector module using PMT photodetectors may have a diameter of 2 meters, while a ring detector module using SiPM photodetectors may have a diameter of 80 centimeters. If the detector module 200 is made to have substantially the same as the size of the conventional detector module, the detector module 200 may have a higher spatial resolution than the conventional detector module.

In some embodiments, a separation layer may be arranged between each adjacent monolithic crystal plates 211. The separation layer between an adjacent monolithic crystal plates 211 may be configured to block scintillation light transmission between the adjacent monolithic crystal plates 211, so that the scintillation light produced by gamma ray interactions in a specific monolithic crystal plate can only travel in the specific monolithic crystal plate (that is, cannot travel through the separation layers to other monolithic crystal plates) and be detected by SiPM photodetector(s) in optical communication with the specific monolithic crystal plate. In this way, a target monolithic crystal plate in which a target gamma ray interaction occurs can be accurately determined, and a spatial resolution of the detector module 200 may be improved. In some embodiments, the separation layer may include a reflective film, a reflective foil, a reflective coating (e.g., a white reflective coating), or any other material that can prevent or substantially prevent light transmission.

In some embodiments, the detector module 200 may further include a light guide (e.g., a light guide 230 as shown in FIGS. 3A-3C) disposed between the semi-monolithic crystal 210 and the photodetector array 220. The light guide may be configured to guide scintillation light from the semi-monolithic crystal 210 to the photodetector array 220. For example, if when scintillation light irradiates the gaps between SiPM photodetectors, the light guide may guide the scintillation light into a corresponding SiPM photodetectors, so that the scintillation light irradiating the gaps between SiPM photodetectors may be also detected. In some embodiments, the light guide may also be used to prevent moisture to protect the semi-monolithic crystal 210. In some embodiments, the light guide may include a frosted glass. In some embodiments, the light guide may include a portion of the semi-monolithic crystal 210. For example, a portion of the semi-monolithic crystal 210 away from the photodetector array 220 may be divided into the plurality of crystal plates 211, and the other portion of the semi-monolithic crystal 210 close to the photodetector array 220 may be a monolithic crystal that is used as the light guide.

In some embodiments, the detector module 200 may be electronically connected to a processing device (e.g., the processing device 120). The processing device may be configured to determine signal intensity information and/or position information relating to gamma ray interactions occur in the semi-monolithic crystal 210. For example, after a target gamma ray interaction occurs in a target monolithic crystal plate among the plurality of monolithic crystal plates 211, the scintillation light produced by target gamma ray interactions can only travel in the target monolithic crystal plate and be detected by one or more target columns of SiPM photodetectors in the photodetector array 220 that are in optical communication with the target monolithic crystal plate. The target column(s) of SiPM photodetectors may convert the detected scintillation light into electrical signals (also referred to as readout signals) and transmit the readout signals to the processing device. In some embodiments, a signal readout sampling rate of each of the plurality of SiPM photodetectors may be in a range from 20 MHz to 150 MHz. In some embodiments, the signal readout sampling rate of each of the plurality of SiPM photodetectors may be in a range from 25 MHz to 120 MHz.

After the readout signals are received from the target column(s) of SiPM photodetectors, the processing device may further determine the signal intensity information and/or the position information of the target gamma ray interaction based on the received readout signals. For example, the processing device may determine a sum of the signal intensities of the received readout signals as the signal intensity of the target gamma ray interaction. Merely by way of example, as shown in FIG. 3A, if the target gamma ray interaction occurs in the monolithic crystal plate 211-1, the processing device may determine a sum of the signal intensities of the readout signals received from the column S4 of SiPM photodetectors as the signal intensity of the target gamma ray interaction. As another example, as shown in FIG. 3A, if the target gamma ray interaction occurs in the monolithic crystal plate 211-2, the processing device may determine a sum of the signal intensities of the readout signals received from the columns S4 and S5 of SiPM photodetectors as the signal intensity of the target gamma ray interaction.

As another example, if the photodetector array 220 may include a plurality of rows arranged side by side along the length direction of the plurality of monolithic crystal plates 211, the processing device may determine a total signal intensity detected by each row of the plurality of rows based on the readout signals. Further, the processing device may determine a signal intensity distribution in the plurality of rows based on the total signal intensity received by each row of the plurality of rows.

In some embodiments, the readout signal received from each SiPM photodetector in the one or more target columns of SiPM photodetectors may be transmitted respectively to the processing device using any suitable circuit, such as an application specific integrated circuit (ASIC). The processing device may determine a sum of the signal intensities of the received readout signals as the signal intensity of the target gamma ray interaction. In some embodiments, the readout signals from the one or more target columns of SiPM photodetectors may be superimposed to generate a combined readout signal, and then the combined readout signal may be transmitted to the processing device. The processing device may designate a signal intensity of the combined readout signal as the signal intensity of the target gamma ray interaction. Transmitting the combined readout signal may reduce a number of channels for signal transmission compared to transmitting each readout signal individually.

In some embodiments, the processing device may be also configured to determine position information of the target gamma ray interaction based on the signal intensity information of the target gamma ray interaction. The position information of the target gamma ray interaction may include position information of the target gamma ray interaction along one or more of the thickness direction, the length direction, and the depth direction of the plurality of monolithic crystal plates 211.

Specifically, after the processing device receives the readout signals from the target column(s) of SiPM photodetectors, the processing device may determine that the target gamma ray interaction occurs in the target monolithic crystal plate based on the corresponding relationship between the SiPM photodetectors and the monolithic crystal plates, that is, the processing device may determine the position information of the target gamma ray interaction along the thickness direction of the plurality of monolithic crystal plates 211. For example, as shown in FIG. 3B, if there is no the light guide 230, and the processing device receives the readout signals from the column S3 of SiPM photodetectors, the processing device may determine that a target gamma ray interaction occurs in the monolithic crystal plate 211-1. As another example, as shown in FIG. 3A, if the processing device receives the readout signals from the column S4 and S5 of SiPM photodetectors, the processing device may determine which monolithic crystal plate a target gamma ray interaction occurs in by looking up a preset table. The preset table may indicate a relationship between a monolithic crystal plate and corresponding column (s) of SiPM photodetectors detected scintillation light produced by a target gamma ray interaction that occurs in the monolithic crystal plate.

Further, the processing device may determine the position information of the target gamma ray interaction in the target monolithic crystal plate (i.e., the position information along the length direction of the monolithic crystal plates 211 and/or the position information along the depth direction of the monolithic crystal plates 211) based on the readout signals. More descriptions for the determination of the position information of the target gamma ray interaction in the target monolithic crystal plate may be found elsewhere in the present disclosure (e.g., FIG. 5 and the descriptions thereof).

As described elsewhere in the present disclosure, if the SiPM photodetectors are used for SPECT imaging, dark currents occur in the SiPM photodetectors will result in that an energy output signal (a sum of all signals detected by the SiPM photodetectors) of the detector module in the SPECT scanner has a non-negligible background noise, which reduces a signal-to-noise ratio of a resulting SPECT image and an energy resolution of the SiPM photodetectors. According to some embodiments of the present disclosure, the crystal in the detector module 200 is the semi-monolithic crystal 210 including the plurality of monolithic crystal plates 211, which is different from the conventional monolithic scintillator. The scintillation light produced by gamma ray interactions in a specific monolithic crystal plate can only travel in the specific monolithic crystal plate and be detected by SiPM photodetector(s) in optical communication with the specific monolithic crystal plate. In this way, only a small number of SiPM photodetectors are capable of detecting the scintillation light of the specific monolithic crystal plate, the background noise caused by the dark currents in the small number of SiPM photodetectors is relatively small, thereby improving the signal-to-noise ratio of a resulting SPECT image and the energy resolution of the SiPM photodetectors. That is, the detector module 200 of the present disclosure can effectively solve the problems of using SiPM photodetectors in SPECT imaging.

In addition, some conventional detector modules (e.g., the analog Anger detector module) includes a PMT photodetector and a monolithic scintillator, which has a low resolution and a relatively big size. The CZT detector module is usually used for high-energy quantitative detection, it may cause relatively great quantitative errors due to distortion. In addition, the cost of CZT detector module is relatively high.

Compared with the conventional detector modules using PMT photodetectors and the monolithic scintillator, the detector module 200 may have an improved spatial resolution because the propagation range of scintillation photons may be smaller due to the use of the semi-monolithic crystal. Moreover, the spatial resolution of the detector module 200 may be further improved by using the SiPM photodetectors with a size smaller than the PMT photodetectors. In addition, the processing device only needs to receive readout signals from the small number of SiPM photodetectors, which only includes a small amount of data, thereby reducing the dead time of the SPECT imaging system during data transmission and improving the accuracy of the counting of the gamma ray interactions and the efficiency of the SPECT imaging.

Compared with the conventional CZT detector module, the cost of the detector module 200 is lower and the spatial resolution is higher. In general, a minimum of the spatial resolution of CZT detector module is 2.46 mm, while the spatial resolution of the detector module 200 may be smaller than or equal to 1.3 mm.

According to some embodiments of the present disclosure, the position information of the target gamma ray interaction (e.g., the position information of the target gamma ray interaction along the thickness direction, the length direction, and the depth direction of the plurality of monolithic crystal plates 211) may be accurately determined due to using the detector module 200, thereby improving the spatial resolution of the SPECT imaging system 100, especially a pinhole imaging system.

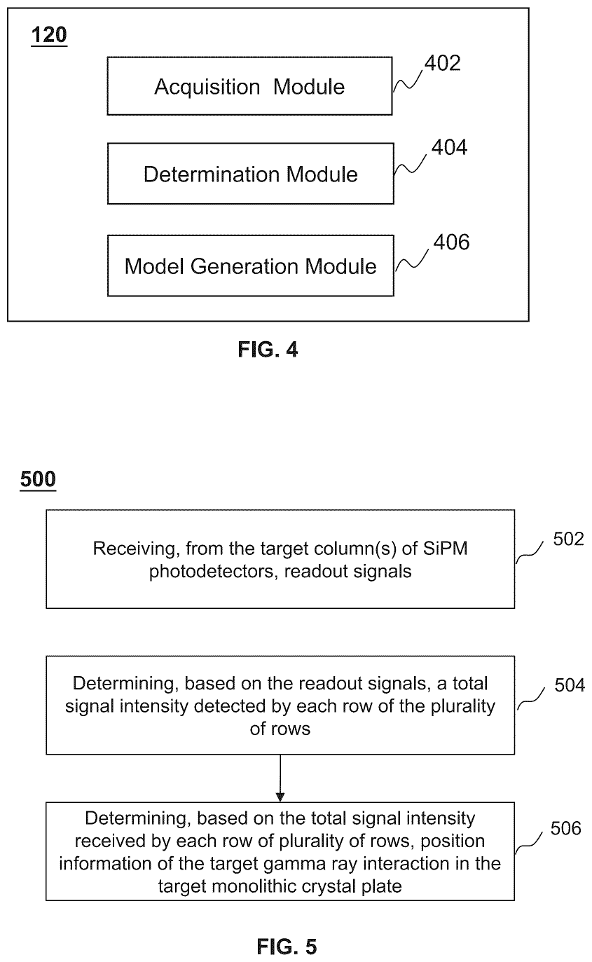

FIG. 4 is a block diagram illustrating an exemplary processing device 120 according to some embodiments of the present disclosure. As illustrated in FIG. 4, the processing device 120 may include an acquisition module 402, a determination module 404, and a model generation module 406. As described in FIG. 1, the SPECT imaging system 100 in the present disclosure may also include multiple processing devices, and the acquisition module 402, the determination module 404, and the model generation module 406 may be components of different processing devices. For example, the acquisition module and the determination module 404 may be components of a processing device 120 A, and the model generation module 406 may be a component of a processing device 120B.

The acquisition module 402 may be configured to information relating to the SPECT imaging system 100. For example, the acquisition module 402 may receive readout signals from target column(s) of SiPM photodetectors. More descriptions regarding the obtaining of the readout signals from target column(s) of SiPM photodetectors may be found elsewhere in the present disclosure. See, e.g., operation 502 in FIG. 5, and relevant descriptions thereof.

The determination module 404 may be configured to determine a total signal intensity detected by each row of the plurality of rows based on the readout signals. The total signal intensity detected by a row may be a total value of the signal intensity detected by each SiPM photodetector in the row. More descriptions regarding the determination of the total signal intensity detected by each row of the plurality of rows based on the readout signals may be found elsewhere in the present disclosure. See, e.g., operation 504 in FIG. 5, and relevant descriptions thereof.

The determination module 404 may be also configured to determine position information of the target gamma ray interaction in the target monolithic crystal plate based on the total signal intensity received by each row of the plurality of rows. More descriptions regarding the determination of the position information of the target gamma ray interaction in the target monolithic crystal plate may be found elsewhere in the present disclosure. See, e.g., operation 506 in FIG. 5, and relevant descriptions thereof.

The model generation module 406 may be configured to generate one or more machine learning models (e.g., a position information determination model) by model training. More descriptions regarding the generation of the position information determination model may be found elsewhere in the present disclosure. See, e.g., FIG. 7, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, any one of the modules may be divided into two or more units. For instance, the acquisition module 402 may be divided into two units configured to acquire different data. In some embodiments, the processing device 120 may include one or more additional modules, such as a storage module (not shown) for storing data.

FIG. 5 is a flowchart illustrating an exemplary process for determining position information of a target gamma ray interaction according to some embodiments of the present disclosure. In some embodiments, process 500 may be executed by the SPECT imaging system 100. For example, the process 500 may be implemented as a set of instructions (e.g., an application) stored in a storage device (e.g., the storage device 130). In some embodiments, the processing device 120 (e.g., one or more modules illustrated in FIG. 4) may execute the set of instructions and may accordingly be directed to perform the process 500. The operations of the illustrated process presented below are intended to be illustrative. In some embodiments, the process 500 may be accomplished with one or more additional operations not described and/or without one or more of the operations discussed. Additionally, the order of the operations of process 500 illustrated in FIG. 5 and described below is not intended to be limiting.

In some embodiments, the target gamma ray interaction may be a gamma ray interaction (e.g., the latest gamma ray interaction) occur in a target monolithic crystal plate in a detector module. For example, the detector module may be the detector module 200 as described in connection with FIG. 2. The detector module 200 may include a semi-monolithic crystal 210 that includes a plurality of monolithic crystal plates 211, and the target monolithic crystal plate may be one of the plurality of monolithic crystal plates 211. The detector module 200 may also include a photodetector array 220. The photodetector array 220 may include a plurality of columns arranged side by side along the thickness direction of the plurality of monolithic crystal plates 211 and a plurality of rows arranged side by side along the length direction of the plurality of monolithic crystal plates 211.

After the target gamma ray interaction occurs in the target monolithic crystal plate, the scintillation light produced by target gamma ray interactions can only travel in the target monolithic crystal plate and be detected by one or more target columns of SiPM photodetectors in the photodetector array 220 that are in optical communication with the target monolithic crystal plate. The target column(s) of SiPM photodetectors may convert the detected scintillation light into electrical signals (also referred to as readout signals) and transmit the readout signals to the processing device 120. After the processing device 120 receives the readout signals from the target column(s) of SiPM photodetectors, the processing device 120 may determine that the target gamma ray interaction occurs in the target monolithic crystal plate based on the corresponding relationship between the SiPM photodetectors and the monolithic crystal plates.

In some embodiments, the target column(s) of SiPM photodetectors may include a plurality of rows arranged along the length direction of the target monolithic crystal plate. That is, the target column(s) of SiPM photodetectors may be a target photodetector array arranged in plurality of rows and one or more columns.

In 502, the processing device 120 (e.g., the acquisition module 402) may receive, from the target column(s) of SiPM photodetectors, readout signals.

In some embodiments, the readout signal received from each SiPM photodetector in the target column(s) of SiPM photodetectors may be transmitted respectively to the processing device 120 using any suitable circuit, such as an ASIC. The processing device 120 may receive the readout signals from the target column(s) of SiPM photodetectors. In some embodiments, the readout signals may be preprocessed (e.g., amplified), and the preprocessed readout signals may be transmitted to the processing device 120 for further processing. For illustration purposes, the following descriptions describe the processing of the readout signals.

In 504, the processing device 120 (e.g., the determination module 404) may determine, based on the readout signals, a total signal intensity detected by each row of the plurality of rows.

The total signal intensity detected by a row may be a total value of the signal intensity detected by each SiPM photodetector in the row. In some embodiments, for each row, the processing device 120 may determine a sum of one or more signal intensities of one or more readout signals from the row, and designate the sum of one or more signal intensities as the total signal intensity from the row.

For example, referring to FIG. 3A, if the target gamma ray interaction occurs in the target monolithic crystal plate 211-1, the target column(s) of SiPM photodetectors may be the column S3 of SiPM photodetectors. The column S3 of SiPM photodetectors may include multiple SiPM photodetectors each belonging to one row of SiPM photodetectors. For each row, the processing device 120 may directly designate the signal intensity of the readout signal received from the SiPM photodetector corresponding to the row as the total signal intensity detected by the row.

As another example, referring to FIG. 3A again, if the target gamma ray interaction occurs in the target monolithic crystal plate 211-2, the target column(s) of SiPM photodetectors may be the columns S4 and S5 of SiPM photodetectors. Each row of the columns S4 and S5 of SiPM photodetectors may include a first SiPM photodetector in the column S4 of SiPM photodetectors and a second SiPM photodetector in the column S5 of SiPM photodetectors. For each row, the processing device 120 may determine a sum of the signal intensities of the readout signals received from the first SiPM photodetector and the second SiPM photodetector corresponding to the row, and designate the sum of the signal intensities of the readout signals received from the first SiPM photodetector and the second SiPM photodetector corresponding to the row as the total signal intensity detected by the row.

In 506, the processing device 120 (e.g., the determination module 404) may determine, based on the total signal intensity received by each row of the plurality of rows, position information of the target gamma ray interaction in the target monolithic crystal plate.

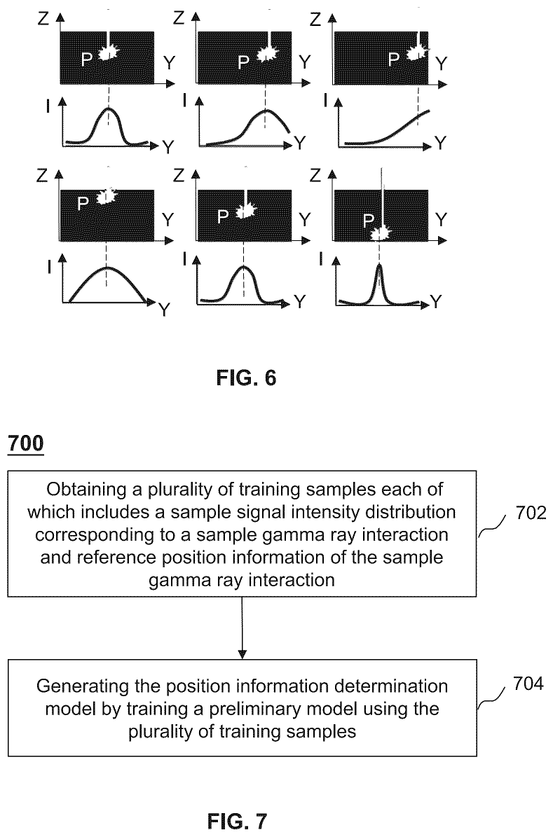

In some embodiments, the processing device 120 may determine a signal intensity distribution (also referred to as a target signal intensity distribution) in the plurality of rows based on the total signal intensity received by each row of the plurality of rows. The signal intensity distribution may indicate total signal intensities corresponding to different rows along the length direction of the target monolithic crystal plate. Merely by way of example, the processing device 120 may establish a two-dimensional (2D) coordinate system including a first coordinate axis parallel to the length direction of the target monolithic crystal plate and a second coordinate axis parallel to a depth direction of the target monolithic crystal plate. For example, the 2D coordinate system may be the same as or similar to a 2D coordinate system including a Y-axis and a I-axis shown in FIG. 6. The second coordinate axis may be configured to denote the total signal intensity received by each row of the plurality of rows. The first coordinate axis may be configured to denote a position of each row of the plurality of rows along the length direction of the target monolithic crystal plate. In some embodiments, the target signal intensity distribution may be represented by a target signal intensity curve in the 2D coordinate system. For example, the processing device 120 may determine a data point corresponding to each row, wherein the coordinate of the data point along the first coordinate axis is determined based on the position of the row along the length direction of the target monolithic crystal plate, and the coordinate of the data point along the second coordinate axis is determined based on the total signal intensity of the row. Further, the processing device 120 may perform a curve fit on a plurality of data points to determine the target signal intensity curve indicative of the target signal intensity distribution.

Further, the processing device 120 may determine at least one of first position information or second position information of the target gamma ray interaction based on the target signal intensity distribution. The first position information may relate to the position of the target gamma ray interaction along the length direction of the target monolithic crystal plate. The second position information may relate to the position of the target gamma ray interaction along the depth direction of the target monolithic crystal plate. For example, the first position information may be denoted by a first coordinate in the Y-axis, and the second position information may be denoted by a second coordinate in the Z-axis.

The position of the target gamma ray interaction along the length direction and the depth direction of the target monolithic crystal plate may affect the target signal intensity distribution (e.g., the shape of the signal intensity curve). For illustration purposes, FIG. 6 provides a schematic diagram illustrating exemplary reference gamma ray interactions and their respective reference signal intensity curves according to some embodiments of the present disclosure. As shown in FIG. 6, the Y-axis denotes a position along the length direction of the target monolithic crystal plate, the Z-axis denotes a position along the depth direction of the target monolithic crystal plate, the I-axis denotes a total signal intensity received by each row of the plurality of rows, and a position point P refers to a position of a reference gamma ray interaction in a reference monolithic crystal plate where the reference gamma ray interaction occurs.

As shown in FIG. 6, for each reference gamma ray interaction, a Y-axis coordinate of the corresponding position point P may substantially correspond to a peak of the corresponding reference signal intensity curves. Accordingly, a position of a gamma ray interaction along a length direction of a monolithic crystal plate where the gamma ray interaction occurs may be determined based on a peak of a signal intensity distribution corresponding to the gamma ray interaction. The processing device 120 may determine the first position information of the target gamma ray interaction according to a peak of the target signal intensity distribution. For example, the processing device 120 may designate a first coordinate of the peak of the target signal intensity distribution as the first position information of the target gamma ray interaction.

As shown in FIG. 6, if the position point P is relatively far from the corresponding incident plane, the peak of the corresponding reference signal intensity curve may have a relatively sharp shape (i.e., the corresponding reference signal intensity curve may have a relatively sharp peak). Accordingly, a position of a gamma ray interaction along a depth direction of a monolithic crystal plate where the gamma ray interaction occurs may be determined based on a shape of a peak of a signal intensity distribution corresponding to the gamma ray interaction. The processing device 120 may determine the second position information of the target gamma ray interaction according to a shape of the peak of the target signal intensity distribution.

In some embodiments, the processing device 120 may obtain multiple reference gamma ray interactions and their corresponding reference signal intensity curves from a storage device (e.g., the storage device 130). Each reference gamma ray interaction may have a known occurrence position. For each of the multiple reference signal intensity curves, the processing device 120 may determine a similarity between the reference signal intensity curve and the target signal intensity curve. Further, the processing device 120 may determine the first position information and/or the second position information based on the known occurrence position of a reference gamma ray interaction whose reference signal intensity curve has the maximum similarity to the target signal intensity curve.

In some determine, the processing device 120 may determine the first position information and/or the second position information by processing the target signal intensity distribution (e.g., the target signal intensity curve) using a position information determination model. The position information determination model may be a trained model (e.g., a machine learning model) configured to receive a signal intensity distribution corresponding to a gamma ray interaction as an input, and output position information of the gamma ray interaction. Merely by way of example, the target signal intensity curve may be input into the position information determination model, and the position information determination model may output the first position information and/or the second position information.

In some embodiments, the position information determination model may include a deep learning model, such as a Deep Neural Network (DNN) model, a Convolutional Neural Network (CNN) model, a Recurrent Neural Network (RNN) model, a Feature Pyramid Network (FPN) model, etc. Exemplary CNN models may include a V-Net model, a U-Net model, a Link-Net model, or the like, or any combination thereof. Since the position information determination model may learn the optimal mechanism for position information determination based on a large amount of data, the position information (e.g., the first position information, the second position information) of the target gamma ray interaction determined using the position information determination model may be relatively accurate.

In some embodiments, the processing device 120 may obtain the position information determination model from one or more components of the SPECT imaging system 100 (e.g., the storage device 130) or an external source via a network. For example, the position information determination model may be previously trained by a computing device (e.g., the processing device 120 or a processing device of a model vendor), and stored in the storage device 130. The processing device 120 may access the storage device 130 and retrieve the position information determination model. In some embodiments, the position information determination model may be generated according to a machine learning algorithm as described elsewhere in this disclosure (e.g., FIG. 4 and the relevant descriptions).

In some embodiments, the position information determination model may be generated by performing process 700 as shown in FIG. 7.

In 702, the processing device 120 (e.g., the model generation module 406) may obtain a plurality of training samples each of which includes a sample signal intensity distribution corresponding to a sample gamma ray interaction and reference position information of the sample gamma ray interaction.

In some embodiments, the plurality of training samples may be acquired based on a same sample SPECT scan using a sample detector module same as or similar to the detector module 200. Alternatively, the plurality of training samples may be acquired based on different sample SPECT scans using the sample detector module or multiple sample detector modules. In some embodiments, the reference position information of the sample gamma ray interaction can be used as a ground truth (also referred to as a label) for model training. In some embodiments, the reference position information of a sample gamma ray interaction may be determined by a user manually. In some embodiments, the reference position information of a sample gamma ray interaction may include at least one of first sample position information or second sample position information of the sample gamma ray interaction. The first sample position information and the second sample position information may be similar to the first position information and the second position information described in FIG. 5, respectively.

In some embodiments, the processing device 120 may obtain a training sample (or a portion thereof) from one or more components of the SPECT imaging system 100 (e.g., the storage device 130) or an external source (e.g., a database of a third-party) via a network.

In 704, the processing device 120 (e.g., the model generation module 406) may generate the position information determination model by training a preliminary model using the plurality of training samples.

The preliminary model refers to a model to be trained. The preliminary model may be of any type of model (e.g., a machine learning model) as described elsewhere in this disclosure (e.g., FIG. 5 and the relevant descriptions). In some embodiments, the processing device 120 may obtain the preliminary model from one or more components of the SPECT imaging system 100 or an external source (e.g., a database of a third-party) via a network. The preliminary model may include a plurality of model parameters. Before training, the model parameters of the preliminary model may have their respective initial values. For example, the processing device 120 may initialize parameter values of the model parameters of the preliminary model.

In some embodiments, the training of the preliminary model may include one or more iterations. For illustration purposes, the implementation of a current iteration is described hereinafter. In some embodiments, a same set or different sets of training samples may be used in different iterations in training the preliminary model. For the convenience of descriptions, a training sample used in the current iteration is referred to as a target training sample.

Merely by way of example, in the current iteration, the processing device 120 may obtain predicted position information of the sample gamma ray interaction of the target training sample based on an intermediate preliminary model. If the current iteration is the first iteration, the intermediate preliminary model may be the preliminary model. If the current iteration is an iteration other than the first iteration, the intermediate preliminary model may be an updated preliminary model generated in a previous iteration. For example, for the target training sample, the processing device 120 may generate a sample model input (e.g., a sample signal intensity distribution corresponding to the target training sample) and input the sample model input into the updated preliminary model. The updated preliminary model may output the predicted position information of the target training sample.

The processing device 140B may further determine a value of a loss function based on the predicted position information of the target training sample. The loss function may be used to measure a discrepancy between a position information predicted by the preliminary model (or the updated preliminary model) in an iteration and the reference position information. Exemplary loss functions may include a focal loss function, a log loss function, a cross-entropy loss, a Dice ratio, or the like.

The processing device 140B may then determine an assessment result of the intermediate preliminary model based on the value of the loss function. The assessment result may indicate whether the intermediate preliminary model is sufficiently trained. For example, the processing device 120 may determine whether a termination condition is satisfied in the current iteration based on the value of the loss function. Exemplary termination conditions may be that the value of the loss function in the current iteration is less than a threshold value, a difference between the values of the loss function obtained in a previous iteration and the current iteration (or among the values of the loss function within a certain number or count of successive iterations) is less than a certain threshold, a maximum number (or count) of iterations has been performed, or the like, or any combination thereof.

In response to determining that the termination condition is not satisfied in the current iteration, the processing device 120 may determine that the intermediate preliminary model is not sufficiently trained, and further update the intermediate preliminary model based on the value of the loss function. Merely by way of example, the processing device 120 may update at least some of the parameter values of the intermediate preliminary model according to, for example, a backpropagation algorithm. The processing device 120 may further perform a next iteration until the termination condition is satisfied. In response to determining that the termination condition is satisfied in the current iteration, the processing device 120 may determine that the intermediate preliminary model is sufficiently trained and terminate the training process. The intermediate preliminary model may be designated as the position information determination model.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.), or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in a baseband or as part of a carrier wave. Such a propagated signal may take any of a variety of forms, including electromagnetic, optical, or the like, or any suitable combination thereof. A computer readable signal medium may be any computer readable medium that is not a computer readable storage medium and that may communicate, propagate, or transport a program for use by or in connection with an instruction-performing system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby, and Groovy, or other programming languages. The program code may execute entirely on the user's computer, partly on the user's computer, as a stand-alone software package, partly on the user's computer, and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A detector module for single photon emission computed tomography (SPECT), comprising:
a semi-monolithic crystal including a plurality of monolithic crystal plates configured to receive gamma rays, the plurality of monolithic crystal plates being arranged side by side along a thickness direction of the plurality of monolithic crystal plates; and
a plurality of silicon photomultiplier (SiPM) photodetectors forming a photodetector array, the photodetector array including a plurality of columns arranged side by side along the thickness direction of the plurality of monolithic crystal plates, wherein
for each of the plurality of monolithic crystal plates, the monolithic crystal plate is in optical communication with one or more columns of SiPM photodetectors in the photodetector array, and the one or more columns of SiPM photodetectors are configured to detect scintillation light produced by gamma ray interactions in the monolithic crystal plate.

2. The detector module of claim 1, wherein the detector module is electronically connected to a processing device, and the processing device is configured to:
receive, from one or more target columns of SiPM photodetectors in the photodetector array that are in optical communication with a target monolithic crystal plate among the plurality of monolithic crystal plates, readout signals; and
determine, based on the readout signals, that a target gamma ray interaction occurs in the target monolithic crystal plate.

3. The detector module of claim 2, wherein the one or more target columns of SiPM photodetectors include a plurality of rows arranged along a length direction of the target monolithic crystal plate, and the processing device is further configured to:
determine, based on the readout signals, a total signal intensity detected by each row of the plurality of rows; and
determine, based on the total signal intensity received by each row of the plurality of rows, position information of the target gamma ray interaction in the target monolithic crystal plate.

4. The detector module of claim 3, wherein to determine, based on the total signal intensity received by each row of the plurality of rows, position information of the target gamma ray interaction in the target monolithic crystal plate, the processing device is configured to:
determine, based on the total signal intensity received by each row of the plurality of rows, a signal intensity distribution in the plurality of rows; and
determine, based on the signal intensity distribution, at least one of first position information or second position information, the first position information relating to the position of the target gamma ray interaction along the length direction of the target monolithic crystal plate, and the second position information relating to the position of the target gamma ray interaction along a depth direction of the target monolithic crystal plate.

5. The detector module of claim 4, wherein to determine, based on the signal intensity distribution, at least one of first position information or second position information, the processing device is configured to:
determine the at least one of the first position information or the second position information by processing the signal intensity distribution using a position information determination model.

6. The detector module of claim 5, wherein the position information determination model is generated by a model training process comprising:
obtaining a plurality of training samples each of which includes a sample signal intensity distribution corresponding to a sample gamma ray interaction and reference position information of the sample gamma ray interaction; and
generating the position information determination model by training a preliminary model using the plurality of training samples.

7. The detector module of claim 1, wherein the detector module further includes a light guide disposed between the semi-monolithic crystal and the photodetector array, and configured to guide the scintillation light from the semi-monolithic crystal to the photodetector array.

8. The detector module of claim 1, wherein a material of the semi-monolithic crystal includes at least one of cesium iodide (CsI) or sodium iodide (NaI).

9. The detector module of claim 1, wherein a thickness of each of at least a portion of the plurality of monolithic crystal plates is smaller than 1.3 millimeters.

10. The detector module of claim 1, wherein a distance between adjacent monolithic crystal plates among the plurality of monolithic crystal plates is smaller than 0.1 millimeters.

11. The detector module of claim 1, wherein a size of each column in the photodetector array along the thickness direction of the plurality of monolithic crystal plates is greater than a thickness of the monolithic crystal plate being in optical communication with the each column.

12. The detector module of claim 1, wherein a signal readout sampling rate of each of the plurality of SiPM photodetectors is in a range from 20 MHz to 150 MHz.

13. The detector module of claim 1, wherein a range of energy that the detector module focuses on is a range from 20 kev to 1000 kev.

14. A single photon emission computed tomography (SPECT) device, comprising:
a detector module configured to detect photons, wherein the detector module includes:
a semi-monolithic crystal including a plurality of monolithic crystal plates configured to receive gamma rays, the plurality of monolithic crystal plates being arranged side by side along a thickness direction of the plurality of monolithic crystal plates; and
a plurality of silicon photomultiplier (SiPM) photodetectors forming a photodetector array, the photodetector array including a plurality of columns arranged side by side along the thickness direction of the plurality of monolithic crystal plates, wherein
for each of the plurality of monolithic crystal plates, the monolithic crystal plate is in optical communication with one or more columns of SiPM photodetectors in the photodetector array, and the one or more columns of SiPM photodetectors are configured to detect scintillation light produced by gamma ray interactions in the monolithic crystal plate; and
a collimator configured to limit a range of photons entering the detector module.

15. The SPECT device of claim 14, wherein the detector module is electronically connected to a processing device, and the processing device is configured to:
receive, from one or more target columns of SiPM photodetectors in the photodetector array that are in optical communication with a target monolithic crystal plate among the plurality of monolithic crystal plates, readout signals; and
determine, based on the readout signals, that a target gamma ray interaction occurs in the target monolithic crystal plate.

16. The SPECT device of claim 15, wherein the one or more target columns of SiPM photodetectors include a plurality of rows arranged along a length direction of the target monolithic crystal plate, and the processing device is further configured to:
determine, based on the readout signals, a total signal intensity detected by each row of the plurality of rows; and
determine, based on the total signal intensity received by each row of the plurality of rows, position information of the target gamma ray interaction in the target monolithic crystal plate.

17. The SPECT device of claim 14, wherein to determine, based on the total signal intensity received by each row of the plurality of rows, position information of the target gamma ray interaction in the target monolithic crystal plate, the processing device is configured to:
determine, based on the total signal intensity received by each row of the plurality of rows, a signal intensity distribution in the plurality of rows; and
determine, based on the signal intensity distribution, at least one of first position information or second position information, the first position information relating to the position of the target gamma ray interaction along the length direction of the target monolithic crystal plate, and the second position information relating to the position of the target gamma ray interaction along a depth direction of the target monolithic crystal plate.

18. The SPECT device of claim 17, wherein to determine, based on the signal intensity distribution, at least one of first position information or second position information, the processing device is configured to:
determine the at least one of the first position information or the second position information by processing the signal intensity distribution using a position information determination model.

19. The SPECT device of claim 14, wherein the detector module further includes a light guide disposed between the semi-monolithic crystal and the photodetector array, and configured to guide the scintillation light from the semi-monolithic crystal to the photodetector array.

20. The SPECT device of claim 14, wherein a material of the semi-monolithic crystal includes at least one of cesium iodide (CsI) or sodium iodide (NaI).
